(19) United States
(12) Patent Application Publication
(10) Pub. No.: US 2007/0173935 A1
(43) Pub. Date: Jul. 26, 2007

(54) Nucleus pulposus augmentation pretreatment technique

(76) Inventors: Michael J. O'Neil, West Barnstable, MA (US); Ian Burgess, Barrington, RI (US); Jeffrey K. Sutton, Medway, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/262,232

(22) Filed: Oct. 28, 2005

(51) Int. Cl. A61F 2/44

(52) U.S. Cl. 623/17.11; 606/61

(57) ABSTRACT

This invention is directed to an intra-operative technique to improve attachment and/or prevent expulsion of nucleus pulposus replacement/augmentation devices and/or to seal fissures in the annulus fibrosus of intervertebral discs.

### BACKGROUND OF THE INVENTION

1. Field of the Invention

Inadequate attachment of nucleus pulposus replacement or augmentation devices can allow for migration and/or expulsion of these implants leading to continued disc height loss, irritation of neighboring tissues (including nerve roots), thereby creating additional pain and/or re-operation. This invention is concerned with improving nucleus pulposus replacement or augmentation devices by enhancing the device(s)' adherence or integration within the evacuated intervertebral disc space while also sealing fissures that may exist in the annulus fibrosus to prevent either nucleus pulposus or device extrusion.

2. Related Art

Several current techniques for nucleus pulposus augmentation and replacement utilize preformed devices (balls, spheres, etc. . . .) comprised of elastomers, hydrogels, ceramics, for example. Other techniques employ injection of in-situ curable materials like the Sinux® silicone or Dascor® polyurethane disc augmentation materials. These concepts rely on mechanical fit and/or material interdigitation to minimize the potential of device movement and expulsion.

Nucleus pulposus and annular repair patents have been applied and issued, some including interior liners and membranes and bags for lining the interior surface of a intervertebral disc under repair. These materials are either placed or injected directly into the disc space (sometimes allowing leaks through a compromised annular wall) or injected into a preformed balloon that is inflated within the disc space.

U.S. Pat. No. 6,923,813 discloses the use of balloons to compress bone in vertebral bodies.

U.S. Pat. No. 5,888,220, U.S. Pat. No. 6,248,131, US 20030220649, and US 20030195628 disclose the use of injectable bags filled with in-situ curable polyurethane and disclose that the material may be injected via a separate lumen outside the of the bag following introduction of the bag into the disc space.

US 20030082169 discloses the use of fluent pretreatments for the endplates of an intervertebral disc purported to decalcify the vertebral endplates, improve vascularity of the vertebral endplates, use of enzymes to enhance interdigitation with a nuclear pulposus prothesis device, or use of trypsin to enhance cell migration from the endplate to a replacement scaffold device.

The prior art discloses the use of fluent materials for nucleus pulposus replacement devices in the disc space and as in US 20030082169, to enhance performance of the replacement device through pretreatment of vertebral endplates. The historical art does not disclose the use of in-situ curable balloons and/or the methods to deploy a pretreatment or primer via spray, swab, balloon or balloon transfer methods to enhance securement to the cleared disc space and seal fissures that may exist in the walls of the annulus fibrosus.

### BRIEF DESCRIPTION OF THE DRAWINGS

FIGS. 1a and 1b depict application of disc pretreatment material by spraying.

FIGS. 2a and 2b depict application of the disc pretreatment material by an in-situ formed balloon.

FIGS. 3a and 3b depict application of the disc pretreatment material by a coated balloon.

FIGS. 4a and 4b depict application of the disc pretreatment material by permeable balloon.

### SUMMARY OF THE INVENTION

The invention is generally related to application of pretreatment materials that help secure intervertebral disc augmentation or replacement device and desirably fill or seal fissures that may be present in the walls of the annulus fibrosus of the intervertebral disc.

Thus in one embodiment, the invention relates to a method for treatment of a degenerative intervertebral disc comprising the steps of:

a) forming an intervertebral disc space through the excision of all or a portion of the nucleus pulposus of the intervertebral disc; and

b) applying a pretreatment material to treat the surfaces of the intervertebral disc space wherein the pretreatment material enhances attachment of an intervertebral replacement or augmentation device to the treated surfaces of the intervertebral disc space.

Other specific embodiments of the invention relate to the application of the pretreatment material such as by injection with gases, swabbing with a flexible device, in-situ balloon creation by dispensing a curable gel through a cannula and inflating into a balloon that cures during/upon contact with the inner annulus (in-situ formed balloon), or a pre-coated balloon that is inflated in the disc space to transfer the primer from the balloon to the inner annulus fibrosus walls and surfaces of vertebral endplates. Other means include flushing or by a lavage with a solution of the pretreatment material, or by inflating a flexible, permeable balloon with a solution of the pretreatment material, thus allowing the solution to flow through the pores of the balloon and to contact the inner annulus fibrosus walls and vertebral endplates.

### DETAILED DESCRIPTION OF PREFERRED EMBODIMENTS OF THE INVENTION

Following discectomy, nucleotomy, or any other method to excise all or a portion of the nucleus pulposus, a primer or pretreatment material is inserted into the surgically created nucleus defect to provide for nucleus replacement device securement and to seal any fissures in the annular wall of the annulus fibrosus. Several methods are disclosed to enable deployment of the primer as hereinafter disclosed. In certain embodiments, it may be desirable to dehydrate the cleared cavity formed by removal of the nucleus pulposus, and in particular the inner walls of the annulus fibrosus or cartilaginous end plates, in order to facilitate adherence of the pretreatment material to the inside surface of the evacuated disc cavity. Of course, the degree of dehydration should be controlled such as not to damage tissue and have the aim of dehydrating so as not to cause harm to tissue yet condition the tissue surface for improved adherence of the pretreatment material. Such dehydration may be accomplished by passing a gas over the surfaces. Such gases may be heated and comprises such gases as air, CO2, nitrogen, or other inert gases. Also pretreatment may comprise multiple pretreatment steps.

A primer or pretreatment composition is applied to the disc space to accomplish any or all of the following: (i) enhance nucleus pulposus augmentation or replacement device securement in the intervertebral space from annular wall defects; (ii) seal the inner annulus; or (iii) prevent nucleus pulposus or device extrusion through annular fissures thereby assisting in maintaining disc height. The primer or pretreatment compositions are preferably in-situ curable materials produced from biologically and chemically compatible materials that facilitate attachment to annular tissue or vertebral endplate as well as the nuclear devices.

Examples of suitable pretreatment materials include platelet rich plasma (PRP), fibrin glues, cyanoacrylates, hydrogels, adhesive polymers, polyanhydrides, amino acids, peptides, and/or chemical formulations with functional side and/or end groups that facilitate attachment to the nucleus pulposus replacement material and/or the intervertebral disc tissue/annulus, e.g., organofunctional silanes, Glutaraldhyde, photo curable materials such as N,N′-bis(3-diazopyruvoyl)-2,2′-(ethylenedioxy)bis-(ethylamine), (DPD)(as disclosed in U.S. Pat. No. 6,783,539, the disclosure of which is hereby incorporated by reference) and 1,8-naphthalimide dyes, or other materials proven to adhere or crosslink to the native intervertebral disc tissues and the nucleus replacement or augmentation device of selection.

The pretreatment material composition is desirably customized based upon the specific chemistry of the nucleus replacement material(s). For instance, a silicone injection used to replace or augment the removed nucleus pulposus can utilize a primer that has been developed for silicone securement based upon current art to enhance silicone attachment to alternate or collagen based substrates. Typically such a primer will include a portion of the base polymer (silane) with added functional end groups for chemical attachment or fillers for mechanical attachment of the nuclear replacement silicone. The primers can also be loaded with fillers and/or chemical agents that have the function of changing the surface morphology and/or surface energy of the inner disc space to further enhance device attachment.

The term fillers, as used herein, is intended to describe additives that may be compounded with the pretreatment material for specific capabilities, including but not limited to optimize viscosity, strength, or imaging. They can be common polymers, carbon fibers, carbon nanotubes, or metallic fillers selected from (Si, Ca, BaSO4, Ca SO4, CaCO3, hydroxyapatite, Ti, Ta, TiO2, etc. . . .), or they can be biologically based.

When desiring to secure nucleus pulposus replacement or augmentation materials while sealing fissures in the walls of the annulus fibrosus, preferred primers include, but are not limited to, materials that have at least some adhesive characteristics such as PRP, fibrin glues, cyanoacrylates, adhesive polymers, polyanhydrides, and/or chemical formulations with functional side and/or end groups that facilitate attachment to the nuclear replacement material and/or the intervertebral disc tissue/annulus, e.g. glutaraldhyde, N,N′-bis(3-diazopyruvoyl)-2,2′-(ethylenedioxy)bis-(ethylamine), 1,8-naphthalimide dyes, organofunctional silanes such as diacetoxymethylvinylsilane, diacetoxymethylallylsilane, triacetoxyvinylsilane, triacetoxyallylsilane, triacetoxyoctenesilane, triacetoxy-5,7octadienesilane, and the like.

The pretreatment materials can be premixed or mixed/incorporated (i.e., preformed or intra-operatively prepared) with the adhesives, fillers, other materials directly before introducing the mixture into the intervertebral disc space.

An exemplary primer/adhesive material combination would be the use of silicone as the adhesive material and an organofunctional silane as the primer. More specifically, since the collagen of the annulus is rich in hydroxyproline, this hydroxyl group makes an ideal target for attachment of a primer/cross-linking agent. The hydrolysis of alkoxy-silane bonds creates a reactive species capable of displacing hydrogen from hydroxyl groups, and acetoxy-silane groups would be preferred for biocompatibility of the liberated alcohol (ethanol) therefrom. The silane atom could be mono-, di-, or tri-substituted with acetoxy groups, with the fourth silane bonding site occupied by an unsaturated hydrocarbon, e.g. vinyl, allyl, or others up to about fifteen carbons, capable of bonding to/with the silicone polymer. The use of methoxy groups commonly employed in organofunctional silanes would be avoided, due to the toxicity of the liberated methanol. Thus, exemplary primers include dimethylacetoxyvinylsilane, dimethylacetoxyallylsilane, diacetoxymethylvinylsilane, diacetoxymethylallylsilane, triacetoxyvinylsilane, triacetoxyallylsilane, methylethylacetoxyvinylsilane, methylethylacetoxyallylsilane, triacetoxyoctenesilane, triacetoxy-5,7octadienesilane, and the like.

The methods of applying the primers or pretreatment materials include injection with gases, swabbing with a flexible device, in-situ balloon creation by dispensing a curable gel through a cannula and inflating into a balloon that cures during/upon contact with the inner annulus fibrosus wall and vertebral endplates (in-situ formed balloon), or a pre-coated balloon that is inflated in the disc space to transfer the pretreatment material from the balloon to the inner walls of the annulus fibrosus and intervertebral endplates. Other means include flushing or lavage with a solution of the pretreatment material, or by inflating a flexible, permeable balloon with a solution of the pretreatment material, thus allowing the solution to flow through the pores of the balloon and to contact the inner annulus fibrosus walls and vertebral endplates. Further explanation of these methods follow with reference to the figures as indicated.

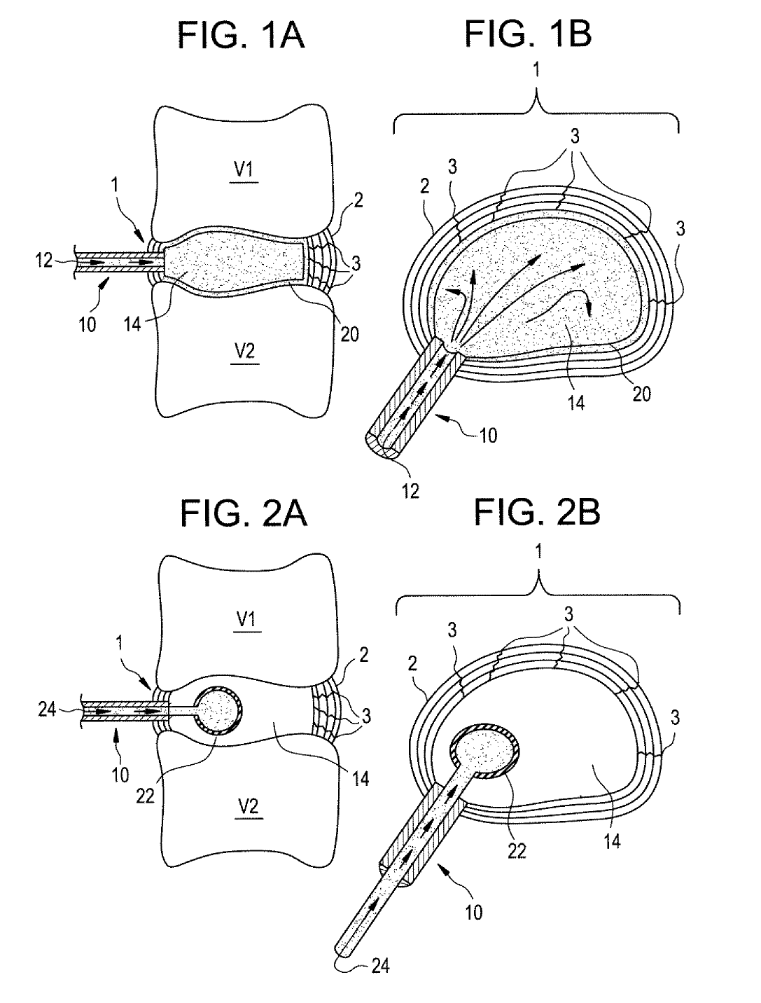

FIGS. 1a and 1b describe application of the pretreatment material by spray including aspiration and the like. FIG. 1b is a cross-sectional, top view of FIG. 1a. (Please note that in all subsequent descriptions of the figures, the “b” version of the figure represents the cross-sectional, top view of the “a” version of the figure). In this method pretreatment material 12 is dispensed into the intervertebral disc space 14 of disc 1. The pretreatment material 12 can be in the form of an aspirate that is mixed with gas (es). Material 12 is preferably atomized and sprayed against the inner annular wall of the annulus fibrosus 2 to form film 20 and desirably to fill and seal the inner annular wall of annulus fibrosus 2, the endplates of vertebral bodies V1 and V2, and any fissures 3 (defects/tears) as well as enable nuclear device attachment. The aspirate 12 can be either physically attached to the intervertebral disc tissue via mechanical interdigitation or through chemical attachment to the intervertebral tissue. The nucleus pulposus replacement or augmentation material can be placed or injected into the intervertebral disc space before the aspirate 12 dries and/or cures. Attachment of the pretreatment material 12 to the device can be mechanical (e.g., by tissue interdigitation) and/or chemical.

Alternately, the pretreatment material may be applied by swabbing or by painting means. In this embodiment, a swab, a brush, (fabric or polymeric foam) is saturated with the pretreatment material and inserted into the disc space to coat the inner annulus with the pretreatment materials.

Yet another method of application of pretreatment material includes lavage. In this case, a solution of the pretreatment material is flushed into the intervertebral space and subsequently removed, for example, by suction, thereby leaving a film of the pretreatment material on the inner surfaces of the intervertebral disc space. Such method could be static, e.g., fill, soak, and drain, or dynamic, e.g., recirculated, or continuously filled and drained. The removal of pretreatment material by lavage may be through the same tube used to introduce the pretreatment material into the intervertebral disc space or through a separate, secondary channel.

Several other methods of application of the pretreatment material include application through use of balloon techniques.

FIGS. 2a and 2b represent another embodiment of this invention wherein an in-situ formed balloon 22 is used in the form of an expandable viscous gel of pretreatment material (e.g. polyurethanes, silicones, hydrogels, adhesives, etc.) that is dispensed by the internal introduction of air (or gas) 24 to “blow” a balloon shape that conforms to the inner intervertebral disc space 14 and cures in-situ. FIGS. 2a and 2b depict this method in the early stages of the formation of in-situ formed balloon 22, well before it conforms to the inner surfaces of intervertebral disc space 14. This method may also be used to determine cleared intervertebral disc volume via monitoring the volume of gas utilized to inflate the in-situ formed balloon.

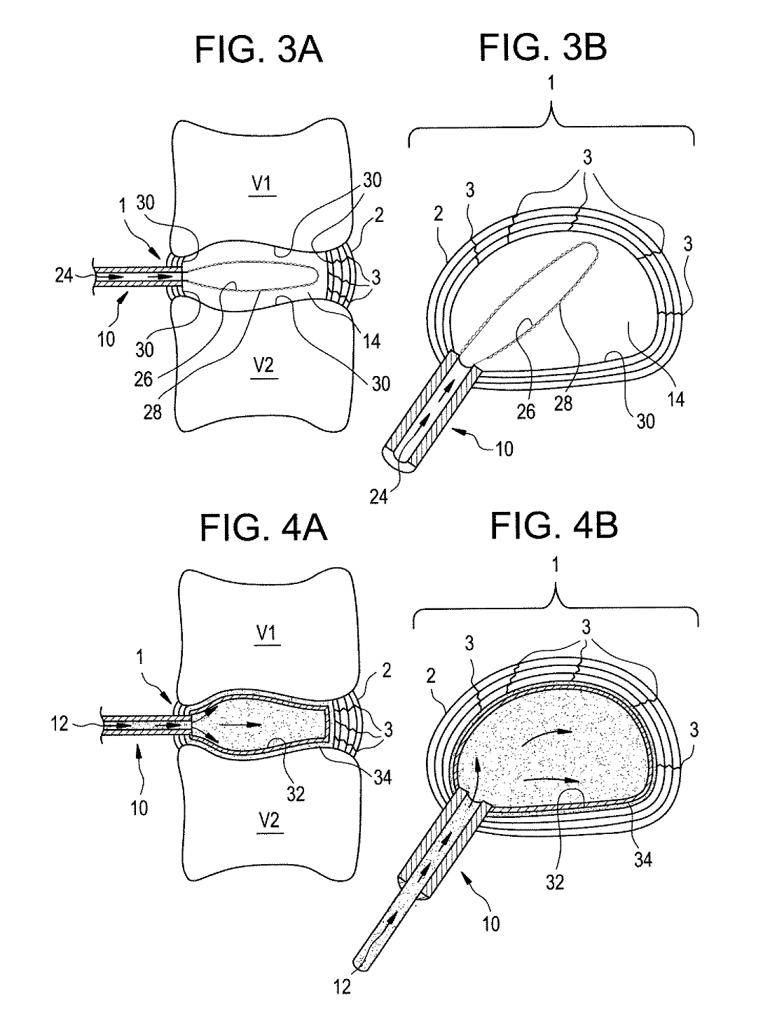

FIGS. 3a and 3b represent yet another embodiment of this invention, wherein a preformed balloon 26 is coated with pretreatment material 28 and inflated by a gas or gases 24 in the intervertebral disc space 14. The pretreatment material(s) 28 are transferred within the cleared intervertebral disc space 14 via contact transfer pressure of the inflated balloon 28 with inner surfaces 30 of intervertebral disc space 14. This method may also be used to determine cleared intervertebral disc volume via monitoring the volume of gas utilized to inflate the balloon.

FIGS. 4a and 4b depict yet a further embodiment of this invention wherein, a flexible, permeable balloon 32 capable of conforming to the contour of the intervertebral disc space and having a porous nature to the balloon material 32, either by nature of it's macromolecular structure or by deliberately formed holes therein, e.g. laser drilled, thereby allowing transfer of pretreatment material 12 through the balloon 32 to the inner surfaces of the intervertebral disc space to form film 34 and to desirably fill and seal annular fissures 3 that may be present in annulus fibrosus 2. In one application of this embodiment, the balloon 32 could be inflated with a solution of the pretreatment material 12 and allowed to “soak” for a period of time, thereby allowing the pretreatment material 12 to exit the balloon 32 through the pores and contact the inner surfaces of the intervertebral disc space as shown for example by film 34.

It should be understood that the foregoing disclosure and description of the present invention are illustrative and explanatory thereof and various changes in the size, shape and materials as well as in the description of the preferred embodiment may be made without departing from the spirit of the invention.

What is claimed is:

1. A method for treatment of a degenerative intervertebral disc comprising the steps of:
a) forming an intervertebral disc space through the excision of all or a portion of the nucleus pulposus of the intervertebral disc; and
b) applying a pretreatment material to treat the surfaces of the intervertebral disc space wherein the pretreatment material enhances attachment of an intervertebral replacement or augmentation device to the treated surfaces of the intervertebral disc space.

2. The method of claim 1, wherein the step of applying the pretreatment material is by spraying.

3. The method of claim 1, wherein the step of applying the pretreatment material is by swabbing.

4. The method of claim 1, wherein the step of applying the pretreatment material is by lavage.

5. The method of claim 1, wherein the step of applying the pretreatment material is by balloon transfer.

6. The method of claim 1, wherein the step of applying the pretreatment material by in-situ formed balloon made of the pretreatment material.

7. The method of claim 5, wherein the step of applying the pretreatment material by balloon transfer is by a balloon coated with the pretreatment material.

8. The method of claim 5, wherein the step of applying the pretreatment material by balloon transfer is by transfer of pretreatment material through a permeable balloon.

9. The method of claim 1, wherein the pretreatment material is selected from the group consisting of platelet rich plasma (PRP), fibrin glues, cyanoacrylates, hydrogels, adhesive polymers, polyanhydrides, amino acids, peptides, organofunctional silanes, gluteraldhyde, N,N′-bis(3-diazopyruvoyl)-2,2′-(ethylenedioxy)bis-(ethylamine), and 1,8-naphthalimide dyes.

10. The method of claim 9, wherein the pretreatment material is PRP.

11. The method of claim 9, wherein the pretreatment material is a fibrin glue.

12. The method of claim 9, wherein the pretreatment material is a cyanoacrylate.

13. The method of claim 9, wherein the pretreatment material is a hydrogel.

14. The method of claim 9, wherein the pretreatment material is an adhesive polymer.

15. The method of claim 9, wherein the pretreatment material is a polyanhydride.

16. The method of claim 9, wherein the pretreatment material is an amino acid.

17. The method of claim 9, wherein the pretreatment material is a peptide.

18. The method of claim 9, wherein the pretreatment material is glutaraldehyde.

19. The method of claim 9, wherein the pretreatment material is N,N′-bis(3-diazopyruvoyl)-2,2′-(ethylenedioxy)bis-(ethylamine).

20. The method of claim 9, wherein the pretreatment material is a 1,8-naphthalimide dye.

21. The method of claim 9, wherein the pretreatment material is an organofunctional silane.

22. The method of claim 21, wherein the organofunctional silane is selected from the group consisting of dimethylacetoxyvinylsilane, dimethylacetoxyallylsilane, diacetoxymethylvinylsilane, diacetoxymethylallylsilane, triacetoxyvinylsilane, triacetoxyallylsilane, methylethylacetoxyvinylsilane, methylethylacetoxyallylsilane, triacetoxyoctenesilane, and triacetoxy-5,7octadienesilane.

23. The method of claim 1, wherein the pretreatment composition comprises silicone and an organofunctional silane.

24. The method of claim 23, wherein the organofunctional silane is selected from group consisting of dimethylacetoxyvinylsilane, dimethylacetoxyallylsilane, diacetoxymethylvinylsilane, diacetoxymethylallylsilane, triacetoxyvinylsilane, triacetoxyallylsilane, methylethylacetoxyvinylsilane, methylethylacetoxyallylsilane, triacetoxyoctenesilane, and triacetoxy-5,7octadienesilane.

25. A method for treatment of a degenerative intervertebral disc comprising the steps of:
a) forming an intervertebral disc space through the excision of all or a portion of the nucleus pulposus of the intervertebral disc; and
b) applying a pretreatment material to treat the surfaces of the intervertebral disc space wherein the pretreatment material enhances the sealing of fissures within the walls of the annulus fibrosus of the treated surfaces of intervertebral disc space.

26. The method for treatment of a degenerative intervertebral disc comprising the steps of:
a) forming an intervertebral disc space through the excision of all or a portion of the nucleus pulposus of the intervertebral disc;
b) dehydrating the intervertebral disc space; and
c) applying a pretreatment material to treat the surfaces of the intervertebral disc space wherein the pretreatment material enhances attachment of an intervertebral replacement or augmentation device to the treated surfaces of the intervertebral disc space.

27. The method of claim 26, wherein step b) is accomplished by passing a gas over the surfaces of the intervertebral disc space.
